(19) United States
(12) Patent Application Publication
(10) Pub. No.: US 2025/0099033 A1
(43) Pub. Date: Mar. 27, 2025

(54) HIGH THROUGHPUT SCREENING METHOD FOR THE IDENTIFICATION OF PSYCHOACTIVE AGENTS

(71) Applicant: FRIEDRICH MIESCHER INSTITUTE FOR BIOMEDICAL RESEARCH, Basel (CH)

(72) Inventors: Matthias HEINDORF, Allschwil (CH); Georg KELLER, Aarau (CH)

(73) Assignee: FRIEDRICH MIESCHER INSTITUTE FOR BIOMEDICAL RESEARCH, Basel (CH)

(21) Appl. No.: 18/729,693

(22) PCT Filed: Jan. 23, 2023
(86) PCT No.: PCT/IB2023/050530

§ 371 Date: Jul. 17, 2024

(30) Foreign Application Priority Data
Jan. 24, 2022 (EP) 22153051.2

(51) Int. Cl. A61B 5/00; A61B 5/055; A61B 5/11; A61B 5/245; A61B 5/369

(52) U.S. Cl. CPC A61B 5/4845; A61B 5/055; A61B 5/1104; A61B 5/245; A61B 5/369

(57) ABSTRACT

The present invention provides a method of screening for psychoactive agents comprising the steps of a) simultaneously assessing in a subject, the activity of neurons of at least two regions of the deep cortical layers in the absence of a potential psychoactive agent and correlating these activities, b) simultaneously assessing in said subject, the activity of said neurons of said at least two regions of the deep cortical layers in the presence of the potential psychoactive agent and correlating said activities, and c) comparing the correlations obtained in step a) and b), wherein any significant change in the correlation obtained in step b) as compared to the correlation obtained in step a) is indicative of a potential psychoactive activity of said potential psychoactive agent.

### FIELD OF THE INVENTION

The present invention provides a new method of identifying psychoactive agents by measuring and comparing activities in deep cortical layers.

### BACKGROUND OF THE INVENTION

The treatment of disorders of the mind such as schizophrenia with antipsychotic medications is hindered by side-effects and complications that cause considerable distress and result in low adherence to medications. So-called typical antipsychotics bind with high affinity to the D2 dopamine receptor (DRD2) and a sizable fraction of schizophrenic patients respond poorly to treatment with these medications.

While the atypical medications represent a significant advance in the treatment of psychosis, there are no guidelines to match patients to medication type to both maximize clinical improvements and to minimize the likelihood of harmful adverse effects, such as extrapyramidal movement symptoms and diabetes.

Schizophrenia is a mental disorder characterized by abnormalities in the perception of reality, affecting roughly 1% of the world's population irrespective of ethnicity or geography. Schizophrenia is a complex and poorly understood disorder, likely caused by a range of environmental and genetic factors. There are no known cures for schizophrenia. However, treatment with antipsychotic medications can alleviate some of the symptoms associated with schizophrenia.

Symptoms of schizophrenia are divided into three broad categories: positive, negative and cognitive symptoms. Positive symptoms comprise outward manifestations of psychosis and can include thought disorders, delusions, or auditory hallucinations. Negative symptoms comprise the loss or the pronounced reduction of normal traits or abilities, such as flat or blunted affect and emotion, loss or inability to speak, inability to experience pleasure, lack of motivation or social isolation. Cognitive symptoms materialize as inattention, or the ability to plan and organize.

As described above, antipsychotic medications that are used to treat symptoms of schizophrenia can be divided into two broad classes: typical and atypical antipsychotics. Typical antipsychotic medications, first identified in the 1950s, are generally thought to be more effective in treating the positive symptoms of schizophrenia, but not the negative or cognitive symptoms. Atypical antipsychotics, on the other hand, are effective in treating all three symptoms of schizophrenia. Unfortunately, both typical and atypical antipsychotics have undesirable side effects. For example, prolonged treatment with typical antipsychotics may lead to tardive dyskinesia, tremors, restlessness, rigidity, and muscle spasms (while failing to treat the negative and cognitive symptoms of schizophrenia). Side effects of atypical antipsychotics include agranulocytosis, weight gain, diabetes and high cholesterol. Because of the prevalence and the social costs associated with diseases such as schizophrenia, there is a need to identify new neuroleptic compounds.

### SUMMARY OF THE INVENTION

By analyzing sensory feedback in cortical layers, the present inventors have found that their results could be used for identifying psychoactive agents.

The present invention thus relates to a method of screening for psychoactive agents comprising the steps of a) simultaneously assessing in a subject the activity of neurons of at least two regions of the deep cortical layers in the absence of a potential psychoactive agent and correlating these activities, b) simultaneously assessing in said subject, the activity of said neurons of said at least two regions of the deep cortical layers in the presence of the potential psychoactive agent and correlating said activities, and c) comparing the correlations obtained in step a) and b), wherein any significant change in the correlation obtained in step b) as compared to the correlation obtained in step a) is indicative of a potential psychoactive activity of said potential psychoactive agent. The neurons of deep cortical layers can be neurons of cortical layers 5 and/or 6. In some embodiments, the second region is in an anatomically different area of the brain as compared to the first region. In some embodiments, the activities of six different regions of the brain are measured in the presence and in the absence of the potential psychoactive agent. In some embodiments, the activities of the neurons are measured while the subject is performing a task, for instance locomotion. In the methods of the invention, a decrease in correlation can be indicative of an antipsychotic activity of said potential psychoactive agent, whereas an increase in correlation can be indicative of a propsychotic activity of said potential psychoactive agent. The activity of the neurons can be assessed using any suitable method known to the skilled person, for instance, using EEG, MRI, fMRI, Magnetoencephalography, widefield calcium imaging, or two photon imaging. In some embodiments, the distance, for instance Euclidean distance, for instance in a top-down view, or the axonal length, between the different brains regions can be used in the assessment of the correlation between the change of activities measures in step a) and b), for instance to normalize the values measured. Suitable neurons that can be used for the invention are neurons with intratelencephalic projection patterns.

The method of the invention can be further improved by adding the steps of d) simultaneously assessing in a subject, the activity of neurons of at least two other regions of the brain that are not regions of the deep cortical layers in the absence of a potential psychoactive agent and correlating these activities, e) simultaneously assessing in said subject, the activity of said neurons of said at least two other regions of the brain that are not regions of the deep cortical layers in the presence of the potential psychoactive agent and correlating said activities, and f) comparing the correlations obtained in step d) and e), wherein the absence of any significant change in the correlation obtained in step e) as compared to the correlation obtained in step d) is indicative of a psychoactive activity of said potential psychoactive agent when there is a significant change in the correlation obtained in step b) as compared to the correlation obtained in step a) is indicative of a potential psychoactive activity of said potential psychoactive agent. Such neurons can be of cortical layers 1, 2 and/or 3.

The present invention also provides a psychoactive agent identified using any of the above methods, as well as a biomarker relying on the use of the correlation between the neuronal activity in deep cortical layers of at least two regions of the brain of a subject as a biomarker in the determination of a psychiatric conditions in said subject, wherein excessive correlation in deep cortical layers while the subject is performing certain tasks can be indicative of a psychiatric conditions in said subject.

### DESCRIPTION OF THE FIGURES

FIG. 1: Antipsychotic drug treatment increased mean activity in Tlx3 positive neurons.
Antipsychotic drug—but not psychostimulant—treatment increased the average activity calculated over all recording sessions in the Tlx3-Cre x Ai148 genetic background that expressed GCaMP6 in layer 5 IT neurons but not in C57BL/6 that expressed GCaMP6 brain wide. Squares are the mean of paired naïve—treatment data and error bars indicate the SEM over 4 C57BL/6, 11 Tlx3-Cre x Ai148 mice that received a single injection of an antipsychotic drug (5 mice Clozapine, 3 mice Aripiprazole, 3 mice Haloperidol), and 3 Tlx3-Cre x Ai148 mice that received a single injection of Amphetamine, respectively. Lines indicate individual mice and shades of gray indicate treatment group (from black to light gray: Clozapine, Aripiprazole, Haloperidol, Amphetamine). *: p<0.05; n.s.: not significant, signed rank test over mice.

FIG. 2: Clozapine reduces activity correlations in dorsal cortex.
(A) Average Pearson's correlation coefficient of activity between individual cortical regions in 4 C57BL/6 mice that expressed GCaMP6 brain wide.
(B) As in A, in the same 4 C57/Bl6 mice, but after a single injection of the antipsychotic drug Clozapine. Clozapine had only a minor effect on correlations of cortical activity.
(C) Average Pearson's correlation coefficient of activity between individual cortical regions in 5 Tlx3-Cre x Ai148 mice that expressed GCaMP6 in layer 5 IT neurons.
(D) As in C, in the same 5 Tlx3 x Ai148 mice but after a single injection of the antipsychotic drug Clozapine. Clozapine strongly reduced correlations of cortical activity in Tlx3 positive layer 5 IT neurons.

FIG. 3: Euclidean distance and activity correlations.

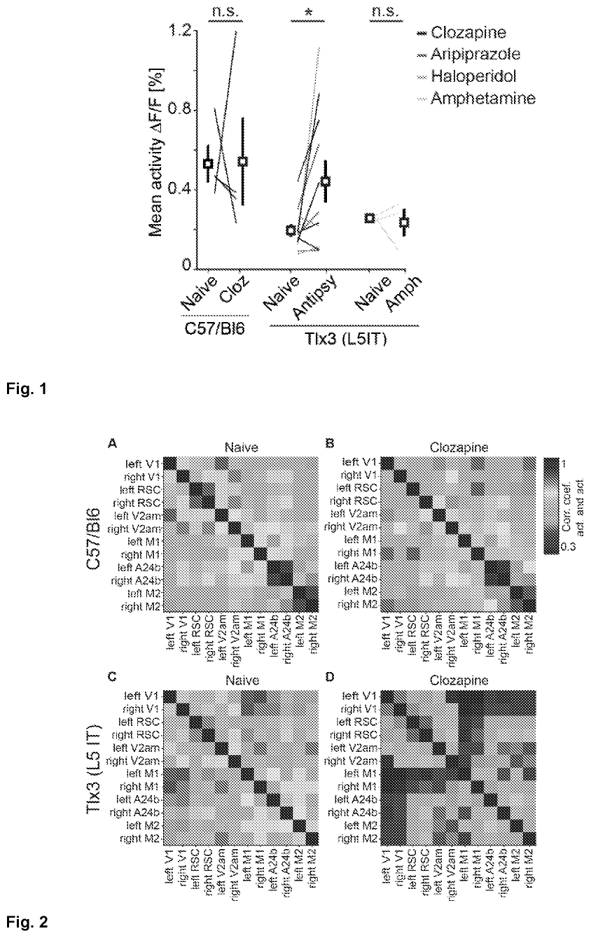

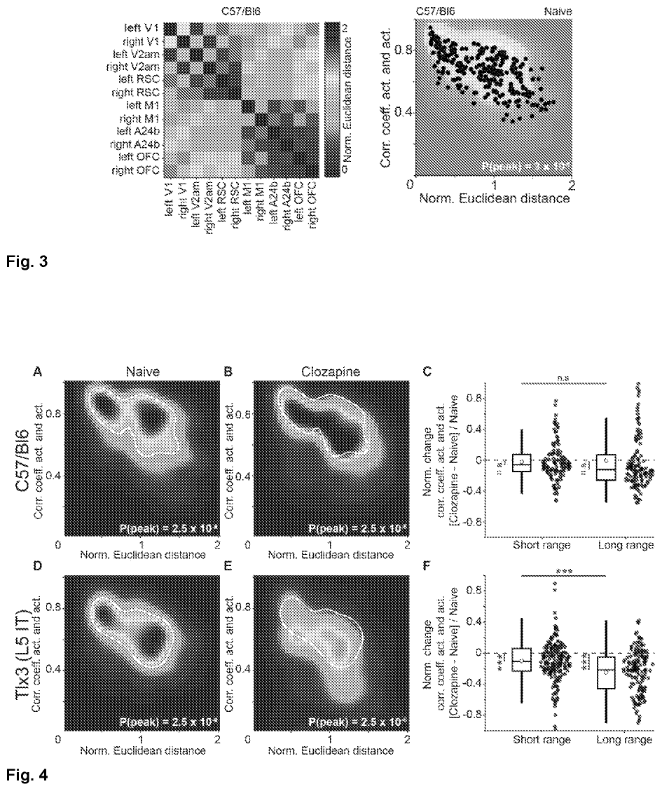

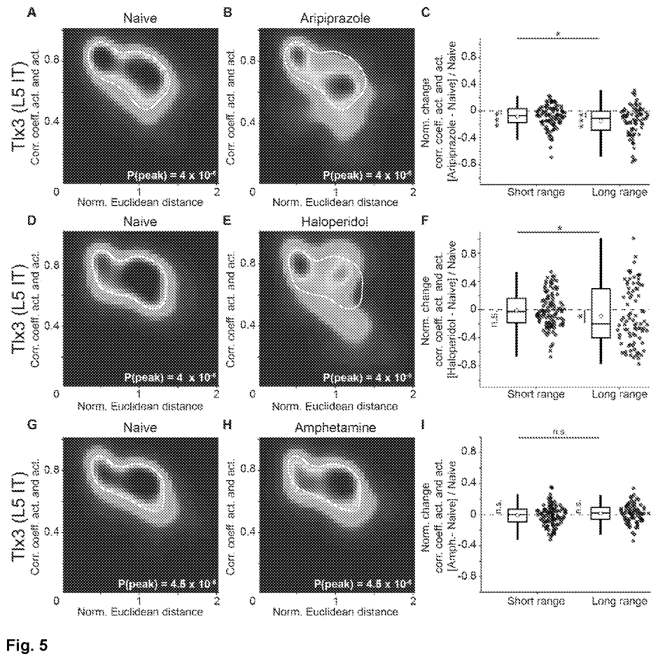

For each pair of dorsal cortex regions, the Euclidean distance between the regions normalized by the Euclidean distance between bregma and lambda anatomical landmarks was calculated (left, data are from 1 example C57/Bl6 mouse that expressed GCaMP6 brain wide). A 40-by-40 2D binned distribution of Pearson's correlation coefficient of activity between pairs of regions and the normalized Euclidean distances between the pairs of regions (right, black dots, data are from the C57/Bl6 example mouse) was calculated to obtain the density plots (right, faded background) shown for the full data sets in FIGS. 2, 4 and 5.

FIG. 4: Tlx3 positive layer 5 IT neurons exhibit the strongest Clozapine induced reduction of long-range correlations.
(A) Density of Pearson's correlation coefficient of activity between individual cortical regions binned to the Euclidean distance between the regions, normalized by the Euclidean distance between bregma and lambda anatomical landmarks, for 4 naive C57/Bl6 mice that expressed GCaMP6 brain wide. P(peak) maps to the maximum value of the color scale. White line indicates a contour drawn to 50% of the maximum bin.
(B) As in A, for the same 4 C57/Bl6 mice after a single injection of the antipsychotic drug Clozapine.
(C) Clozapine induced difference in pairwise region's Pearson's activity correlation coefficient, normalized to the correlation coefficient in naïve state, in 4 C57/Bl6 mice. Data were split into short- and long-range activity correlations. Boxes represent the median and the interquartile range. The open circle indicates the mean of the distribution. The whiskers mark 1.5-fold of the interquartile range. Dots show the individual data points of the full data set (short-range: 122 pairs, long-range: 142 pairs). n.s.: not significant. Statistical comparisons versus no difference were made as t-test. Between-group comparison was a ranksum test. Clozapine did not significantly affect activity correlations in C57/Bl6 mice that expressed GCaMP6 brain wide.
(D) As in A, but for 5 Tlx3-Cre x Ai148 mice that expressed GCaMP6 in layer 5 IT neurons.
(E) As in B, but for 5 Tlx3-Cre x Ai148 mice that expressed GCaMP6 in layer 5 IT neurons.
(F) As in C, but for 5 Tlx3-Cre x Ai148 mice that expressed GCaMP6 in layer 5 IT neurons (short-range: 182 pairs, long-range: 148 pairs). ***: p<0.0005. Clozapine significantly reduced both short- and long-range activity correlations in Tlx3-Cre x Ai148 mice.

FIG. 5: Antipsychotic drugs Aripiprazole and Haloperidol mimicked the decorrelation of dorsal cortex activity observed with Clozapine whereas psychostimulant Amphetamine does not.
(A) Density of Pearson's correlation coefficient of activity between individual cortical regions binned to the Euclidean distance between the regions, normalized by the Euclidean distance between bregma and lambda anatomical landmarks, for 3 Tlx3-Cre x Ai148 mice that express GCaMP6 brain wide. P(peak) maps to the maximum value of the color scale. White line indicates a contour drawn to 50% of the maximum bin.
(B) As in A, for the same 3 Tlx3-Cre x Ai148 mice, after a single injection of the antipsychotic drug Aripiprazole.
(C) Aripiprazole induced difference in pairwise region's Pearson's activity correlation coefficient, normalized to the correlation coefficient in naïve state, in 3 Tlx3-Cre x Ai148 mice. Data were split into short- and long-range activity correlations. Boxes represent the median and the interquartile range. The open circle indicates the mean of the distribution. The whiskers mark 1.5-fold of the interquartile range. Dots show the individual data points of the full data set (short-range: 114 pairs, long-range: 84 pairs). *: p<0.05, ***: p<0.0005. Statistical comparisons versus no difference were made as t-test. Between-group comparison was a ranksum test. Aripiprazole significantly reduced both short- and long-range activity correlations in Tlx3-Cre x Ai148 mice.
(D) As in A, but for 3 different Tlx3-Cre x Ai148 mice.
(E) As in D, for the same 3 Tlx3-Cre x Ai148 mice, but after a single injection of the antipsychotic drug Haloperidol.
(F) As in C, but for the 3 mice that had received a single injection of the antipsychotic drug Haloperidol (short-range: 114 pairs, long-range: 84 pairs). *: p<0.05; n.s.: not significant. Haloperidol reduced long-range activity correlations.
(G) As in A, but for 3 different Tlx3-Cre x Ai148 mice.
(H) As in G, for the same 3 Tlx3-Cre x Ai148 mice, but after a single injection of the psychostimulant Amphetamine.
(I) As in D, but for the 3 mice that had received a single injection of the psychostimulant Amphetamine (short-range: 114 pairs, long-range: 84 pairs). n.s.: not significant. Amphetamine did not affect activity correlations.

### DETAILED DESCRIPTION OF THE INVENTION

By analyzing sensory feedback in cortical layers, the present inventors have found that their results could be used for identifying psychoactive agents.

The present invention thus relates to a method of screening for psychoactive agents comprising the steps of a) simultaneously assessing in a subject the activity of neurons of at least two regions of the deep cortical layers in the absence of a potential psychoactive agent and correlating these activities, b) simultaneously assessing in said subject, the activity of said neurons of said at least two regions of the deep cortical layers in the presence of the potential psychoactive agent and correlating said activities, and c) comparing the correlations obtained in step a) and b), wherein any significant change in the correlation obtained in step b) as compared to the correlation obtained in step a) is indicative of a potential psychoactive activity of said potential psychoactive agent. The neurons of deep cortical layers can be neurons of cortical layers 5 and/or 6. In some embodiments, the second region is in an anatomically different area of the brain as compared to the first region. In some embodiments, the activities of six different regions of the brain are measured in the presence and in the absence of the potential psychoactive agent. In some embodiments, the activities of the neurons are measured while the subject is performing a task, for instance locomotion. In the methods of the invention, a decrease in correlation can be indicative of an antipsychotic activity of said potential psychoactive agent, whereas an increase in correlation can be indicative of a propsychotic activity of said potential psychoactive agent. The activity of the neurons can be assessed using any suitable method known to the skilled person, for instance, using EEG, MRI, fMRI, Magnetoencephalography, widefield calcium imaging, or two photon imaging. In some embodiments, the distance, for instance Euclidean distance, for instance in a top-down view, or the axonal length, between the different brains regions can be used in the assessment of the correlation between the change of activities measures in step a) and b), for instance to normalize the values measured. Suitable neurons that can be used for the invention are neurons with intratelencephalic projection patterns.

The method of the invention can be further improved by adding the steps of d) simultaneously assessing in a subject, the activity of neurons of at least two other regions of the brain that are not regions of the deep cortical layers in the absence of a potential psychoactive agent and correlating these activities, e) simultaneously assessing in said subject, the activity of said neurons of said at least two other regions of the brain that are not regions of the deep cortical layers in the presence of the potential psychoactive agent and correlating said activities, and f) comparing the correlations obtained in step d) and e), wherein the absence of any significant change in the correlation obtained in step e) as compared to the correlation obtained in step d) is indicative of a psychoactive activity of said potential psychoactive agent when there is a significant change in the correlation obtained in step b) as compared to the correlation obtained in step a) is indicative of a potential psychoactive activity of said potential psychoactive agent. Such neurons can be of cortical layers 1, 2 and/or 3.

The present invention also provides a psychoactive agent identified using any of the above methods, as well as a biomarker relying on the use of the correlation between the neuronal activity in deep cortical layers of at least two regions of the brain of a subject as a biomarker in the determination of a psychiatric conditions in said subject, wherein excessive correlation in deep cortical layers while the subject is performing certain tasks can be indicative of a psychiatric conditions in said subject.

These and other aspects of the present invention should be apparent to those skilled in the art, from the teachings herein.

The following definitions are provided to facilitate understanding of certain terms used throughout this specification.

“Antipsychotics”, also known as neuroleptics, are a class of psychotropic medication primarily used to manage symptoms of psychosis (including delusions, hallucinations, paranoia or disordered thought), principally in schizophrenia but also in a range of other psychotic disorders, e.g., bipolar disorder.

A “neuron” or nerve cell is an electrically excitable cell that communicates with other cells via specialized connections called synapses. It is the main component of nervous tissue. Neurons are typically classified into three types based on their function. Sensory neurons respond to stimuli such as touch, sound, or light that affect the cells of the sensory organs, and they send signals to the spinal cord or brain. Motor neurons receive signals from the brain and spinal cord to control everything from muscle contractions to glandular output. Interneurons connect neurons to other neurons within the same region of the brain or spinal cord. A group of connected neurons is called a neural circuit. A typical neuron consists of a cell body (soma), dendrites, and a single axon. The soma is usually compact. The axon and dendrites are filaments that extrude from it. Dendrites typically branch profusely and extend a few hundred micrometres from the soma. At the farthest tip of the axon's branches are axon terminals, where the neuron can transmit a signal across the synapse to another cell.

Neurons may lack dendrites or have no axon. Most neurons receive signals via the dendrites and soma and send out signals down the axon. At most synapses, signals cross from the axon of one neuron to a dendrite of another. However, synapses can connect an axon to another axon or a dendrite to another dendrite. The signalling process is partly electrical and partly chemical. Neurons are electrically excitable, due to maintenance of voltage gradients across their membranes. If the voltage changes by a large enough amount over a short interval, the neuron generates an all-or-nothing electrochemical pulse called an action potential. This potential travels rapidly along the axon and activates synaptic connections as it reaches them. Synaptic signals may be excitatory or inhibitory, increasing or reducing the net voltage that reaches the soma.

“Activity of neurons”, neural activity or neuronal activity encompasses several energy-requiring processes, including presynaptic action potentials, Ca2+ currents, neurotransmitter release and repackaging, and postsynaptic potentials, and can be measured by e.g., recording membrane potential. Functional imaging techniques such as fMRI, PET, and MRS measure the activity of neurons by measuring neurophysiological parameters, i.e., changes in cerebral oxygenation, cerebral metabolism and blood flow, and brain metabolite and neurotransmitter concentrations, respectively.

As used herein, “activity of neurons” can also be assessed by measuring the activity of parts of neurons, e.g. soma, axons or dendrites. Alternatively, it can also be done by measuring the overall activity of a defined region of the brain.

The term “deep cortical layers” refers to layers five and six of the mature cortex. The layered structure of the mature cerebral cortex is formed during development. The first pyramidal neurons generated migrate out of the ventricular zone and subventricular zone, together with reelin-producing Cajal-Retzius neurons, from the preplate. Next, a cohort of neurons migrating into the middle of the preplate divides this transient layer into the superficial marginal zone, which will become layer I of the mature neocortex, and the subplate, forming a middle layer called the cortical plate. These cells will form the deep layers of the mature cortex, layers five and six. Later born neurons migrate radially into the cortical plate past the deep layer neurons and become the upper layers (two to four).

The cerebral cortex, also known as the cerebral mantle, is the outer layer of neural tissue of the cerebrum of the brain in humans and other mammals. The cerebral cortex mostly consists of the six-layered neocortex. It is separated into two cortices, by the longitudinal fissure that divides the cerebrum into the left and right cerebral hemispheres. The two hemispheres are joined beneath the cortex by the corpus callosum. The cerebral cortex plays a key role in attention, perception, awareness, thought, memory, language, and consciousness. In the human brain most of the cerebral cortex is not visible from the outside, but buried in the sulci, and the insular cortex is completely hidden. The major sulci and gyri mark the divisions of the cerebrum into the lobes of the brain. Cortical areas, also called “regions of the cortical layers”, have specific functions such as movement in the motor cortex, and sight in the visual cortex. The whole of the cerebral cortex was divided into 52 different areas, or regions, in an early presentation by Korbinian Brodmann. These areas known as Brodmann areas, are based on their cytoarchitecture but also relate to various functions. An example is Brodmann area 17 which is the primary visual cortex. In more general terms the cortex is typically described as comprising three parts: sensory, motor, and association areas. The sensory areas are the cortical areas that receive and process information from the senses. Parts of the cortex that receive sensory inputs from the thalamus are called primary sensory areas. The senses of vision, hearing, and touch are served by the primary visual cortex, primary auditory cortex and primary somatosensory cortex respectively. In general, the two hemispheres receive information from the opposite (contralateral) side of the body. For example, the right primary somatosensory cortex receives information from the left limbs, and the right visual cortex receives information from the left visual field. The organization of sensory maps in the cortex reflects that of the corresponding sensing organ, in what is known as a topographic map. Neighboring points in the primary visual cortex, for example, correspond to neighboring points in the retina. This topographic map is called a retinotopic map. In the same way, there exists a tonotopic map in the primary auditory cortex and a somatotopic map in the primary sensory cortex. This last topographic map of the body onto the posterior central gyrus has been illustrated as a deformed human representation, the somatosensory homunculus, where the size of different body parts reflects the relative density of their innervation. Areas with much sensory innervation, such as the fingertips and the lips, require more cortical area to process finer sensation. The motor areas are located in both hemispheres of the cortex. The motor areas are very closely related to the control of voluntary movements, especially fine fragmented movements performed by the hand. The right half of the motor area controls the left side of the body, and vice versa.

Examples of “anatomically different” “different regions of the brains”, are, but are not limited to, the different cortical regions, for example, the primary motor cortex, the premotor cortex, the orbitofrontal cortex, the dorsolateral prefrontal cortex, the superior frontal gyrus, the middle frontal gyrus, the inferior frontal gyrus, the parietal lobe, the primary somatosensory cortex (S1), the secondary somatosensory cortex (S2), the posterior parietal cortex, the postcentral gyrus (primary somesthetic area), the primary visual cortex (V1), V2, V3, V4, V5/MT, the lateral occipital gyrus, the primary auditory cortex (A1), the secondary auditory cortex (A2), the inferior temporal cortex, the posterior inferior temporal cortex, the superior temporal gyrus, the middle temporal gyrus, the inferior temporal gyrus, the entorhinal cortex, the perirhinal cortex, the parahippocampal gyrus, the fusiform gyrus, the cingulate cortex, the anterior cingulate, the posterior cingulate and the retrosplenial cortex.

A “significant change” refers to a change that has a p-value, or probability value, i.e. a value describing how likely it is that a change would have occurred by random, of equal or less than 0.05 (typically ≤ 0.05), e.g., p=0.05, p=0.01 or p=0.001.

“Correlation” is a statistical term describing the degree to which two variables move in coordination with one another. If the two variables move in the same direction, then those variables are said to have a positive correlation. If they move in opposite directions, then they have a negative correlation. Correlation, or dependence, is any statistical relationship, whether causal or not, between two random variables or bivariate data. In the broadest sense correlation is any statistical association, though it actually refers to the degree to which a pair of variables are linearly related. Essentially, correlation is the measure of how two or more variables are related to one another. There are several correlation coefficients measuring the degree of correlation. The most common of these is the Pearson correlation coefficient, which is sensitive only to a linear relationship between two variables (which may be present even when one variable is a nonlinear function of the other). Other correlation coefficients—such as Spearman's rank correlation—have been developed to be more robust than Pearson's, that is, more sensitive to nonlinear relationships.

The neocortex is formed of six layers, numbered I to VI, or 1 to 6, from the outermost layer I —near to the pia mater, to the innermost layer VI—near to the underlying white matter. Each cortical layer has a characteristic distribution of different neurons and their connections with other cortical and subcortical regions. There are direct connections between different cortical areas and indirect connections via the thalamus. One of the clearest examples of cortical layering is the line of Gennari in the primary visual cortex. This is a band of whiter tissue that can be observed with the naked eye in the calcarine sulcus of the occipital lobe. The line of Gennari is composed of axons bringing visual information from the thalamus into layer IV of the visual cortex. Staining cross-sections of the cortex to reveal the position of neuronal cell bodies and the intracortical axon tracts allowed neuroanatomists in the early 20th century to produce a detailed description of the laminar structure of the cortex in different species. The work of Korbinian Brodmann (1909) established that the mammalian neocortex is consistently divided into six layers.

Layer I (1) is the molecular layer, and contains few scattered neurons, including GABAergic rosehip neurons. Layer I consists largely of extensions of apical dendritic tufts of pyramidal neurons and horizontally oriented axons, as well as glial cells. Inputs to the apical tufts are thought to be crucial for the feedback interactions in the cerebral cortex involved in associative learning and attention. Layer I across the cerebral cortex mantle receives substantial input from matrix or M-type thalamus cells (in contrast to core or C-type that go to layer IV).

Layer II (2), the external granular layer, contains small pyramidal neurons and numerous stellate neurons.

Layer III (3), the external pyramidal layer, contains predominantly small and medium-size pyramidal neurons, as well as non-pyramidal neurons with vertically oriented intracortical axons; layers I through III are the main target of interhemispheric corticocortical afferents, and layer III is the principal source of corticocortical efferents.

Layer IV (4), the internal granular layer, contains different types of stellate and pyramidal cells, and is the main target of thalamocortical afferents from thalamus type C neurons (core-type) as well as intra-hemispheric corticocortical afferents. The layers above layer IV are also referred to as supragranular layers (layers I-III), whereas the layers below (deep cortical layers) are referred to as infragranular layers (layers V and VI).

Layer V (5), the internal pyramidal layer, contains large pyramidal neurons. Axons from these leave the cortex and connect with subcortical structures including the basal ganglia. In the primary motor cortex of the frontal lobe, layer V contains giant pyramidal cells called Betz cells, whose axons travel through the internal capsule, the brain stem, and the spinal cord forming the corticospinal tract, which is the main pathway for voluntary motor control. Layer VI (6), the polymorphic or multiform layer, contains few large pyramidal neurons and many small spindle-like pyramidal and multiform neurons; layer VI sends efferent fibres to the thalamus, establishing a very precise reciprocal interconnection between the cortex and the thalamus. That is, layer VI neurons from one cortical column connect with thalamus neurons that provide input to the same cortical column. These connections are both excitatory and inhibitory. Neurons send excitatory fibres to neurons in the thalamus and send collaterals to the thalamic reticular nucleus that inhibit these same thalamus neurons or ones adjacent to them.

“IT neurons”, are neurons from the inferior temporal cortex. Inferior Temporal (IT) Cortex is the cerebral cortex on the inferior convexity of the temporal lobe in primates including humans. It is crucial for visual object recognition and is considered to be the final stage in the ventral cortical visual system. It corresponds to cytoarchitecture Areas 20 and 21 (in Brodmans's terminology) and Area TE (in von Economo's). In humans it consists of the middle and inferior temporal gyri. Layer 5 pyramidal neurons of the vM1 are known to include two morphologic groups, intratelencephalic (IT) neurons and pyramidal tract (PT) neurons. A “psychoactive agent”, psychoactive drug, psychopharmaceutical, psychoactive substance, or psychotropic drug, is a chemical substance that changes nervous system function and results in alterations in perception, mood, consciousness, cognition, or behaviour. Such an agent may be used medically; recreationally; to purposefully improve performance or alter one's consciousness; as entheogens for ritual, spiritual, or shamanic purposes; or for research. Some categories of psychoactive drugs, which have therapeutic value, are prescribed by physicians and other healthcare practitioners. Examples include anaesthetics, analgesics, anticonvulsant and antiparkinsonian drugs as well as medications used to treat neuropsychiatric disorders, such as antidepressants, anxiolytics, antipsychotics, and stimulant medications. Psychoactive substances often bring about subjective (although these may be objectively observed) changes in consciousness and mood that the user may find rewarding and pleasant (e.g., euphoria or a sense of relaxation) or advantageous in an objectively observable or measurable way (e.g. increased alertness). Substances which are rewarding and thus positively reinforcing have the potential to induce a state of addiction—compulsive drug use despite negative consequences. In addition, sustained use of some substances may produce physical or psychological dependence or both, associated with somatic or psychological-emotional withdrawal states respectively. Drug rehabilitation attempts to reduce addiction, through a combination of psychotherapy, support groups, and other psychoactive substances. Conversely, certain psychoactive drugs may be so unpleasant that the person will never use the substance again. This is especially true of certain deliriants (e.g. Jimson weed), powerful dissociatives (e.g. PCP, ketamine), and classic psychedelics (e.g. LSD, psilocybin), in the form of a “bad trip”.

A “psychotropic agent”, psychiatric or psychotropic medication is a psychoactive drug taken to exert an effect on the chemical makeup of the brain and nervous system. Thus, these medications are used to treat mental illnesses. These medications are typically made of synthetic chemical compounds and are usually prescribed in psychiatric settings, potentially involuntarily during commitment.

“Antipsychotic agents”, antipsychotics, or neuroleptics, are a class of psychotropic medication primarily used to manage psychosis (including delusions, hallucinations, paranoia or disordered thought), principally in schizophrenia but also in a range of other psychotic disorders. They are also the mainstay together with mood stabilizers in the treatment of bipolar disorder. First-generation antipsychotics, known as typical antipsychotics, were first introduced in the 1950s, and others were developed until the early 1970s. Second-generation drugs, known as atypical antipsychotics, were introduced firstly with clozapine in the early 1970s followed by others. Both generations of medication block receptors in the brain for dopamine, but atypicals tend to act on serotonin receptors as well

“Propsychotic agents”, are agents that promotes psychosis. Examples are Dizocilpine (INN), also known as MK-801, associated with several effects, including cognitive disruption and psychotic-spectrum reactions and is a tool used in research in creating animal models of schizophrenia, or 2,5-Dimethoxy-4-iodoamphetamine (DOI), a psychedelic drug and a substituted amphetamine.

Those skilled in the art are familiar with appropriate dosage for patients being treated with medications that e.g., act at the D2 dopamine receptor, including what is meant by “high dose” and “low dose” antipsychotics. Typically, a low dose treatment with risperidone, 5 for example, would be in the range of 2-4 mg/day for an adult human patient. A high dose would be 6 mg/day or more. As a guide, one can compare differing medications to the chlorpromazine equivalent per kilogram of body weight (CPZEK). For example, one mg risperidone is equipotent to 100 mg chlorpromazine, 100 mg thioridazine, or 2 mg haloperidol.

“Performing a task” means that the subject has to perform an activity while the measurements of the brain activities are taking place. Performing an activity will usually provide sensory input to the brain, feedback, resulting from the motor output sent by the brain.

“EEG” or Electroencephalography is a method to record an electrogram of the electrical activity on the scalp. EEG has been shown to represent the macroscopic activity of the surface layer of the brain underneath. It is typically non-invasive, with the electrodes placed along the scalp. Electrocorticography, involving invasive electrodes, is sometimes called intracranial EEG. EEG measures voltage fluctuations resulting from ionic current within the neurons of the brain. Clinically, EEG refers to the recording of the brain's spontaneous electrical activity over a period of time, as recorded from multiple electrodes placed on the scalp. EEG is one of the few mobile techniques available and offers millisecond-range temporal resolution.

“MRI” or Magnetic resonance imaging is a medical imaging technique used in radiology to form pictures of the anatomy and the physiological processes of the body. This technique uses strong magnetic fields, magnetic field gradients, and radio waves to generate images of the organs in the body. MRI was originally called NMRI (nuclear magnetic resonance imaging).

“fMRI”, functional magnetic resonance imaging, or functional MRI, measures brain activity by detecting changes associated with blood flow. This technique relies on the fact that cerebral blood flow and neuronal activation are coupled. When an area of the brain is in use, blood flow to that region also increases.

“Magnetoencephalography” (MEG) is a functional neuroimaging technique for mapping brain activity by recording magnetic fields produced by electrical currents occurring naturally in the brain, using very sensitive magnetometers.

Calcium imaging is a microscopy technique to optically measure the calcium (Ca2+) concentration of an isolated cell, tissue or medium. Calcium imaging takes advantage of calcium indicators, fluorescent molecules that respond to the binding of Ca2+ions by changing their fluorescence. Two main classes of calcium indicators exist: chemical indicators and genetically encoded calcium indicators (GECI). In neurons, electrical activity is always accompanied by an influx of Ca2+ions. Thus, calcium imaging can be used to approximate the activity in hundreds of neurons in cell culture or in living animals.

Any microscope technique where the entire sample is exposed to light is known as ‘widefield’ imaging. The counterpart to widefield is confocal, where pinholes are used to block most of the light to and from the sample.

“Widefield calcium imaging” allows to simultaneously observe neuronal activity across all cortical areas and, by measuring intracellular calcium levels, measures a signal tightly related to neuronal activity.

“Two photon imaging”, or two-photon excitation microscopy (TPEF or 2PEF) is a fluorescence imaging technique that allows imaging of living tissue up to about one millimeter in thickness. Unlike traditional fluorescence microscopy, in which the excitation wavelength is shorter than the emission wavelength, two-photon excitation requires simultaneous excitation by two photons with longer wavelength than the emitted light. Two-photon excitation microscopy typically uses near-infrared (NIR) excitation light which can also excite fluorescent dyes. Two-photon excitation can be a superior alternative to confocal microscopy due to its deeper tissue penetration, efficient light detection, and reduced photobleaching. As used in the claims, the “distance” refers to a numerical measurement of how far apart objects or points are. The “Euclidean distance” distance between two points in Euclidean space is the length of a line segment between the two points. It can be calculated from the Cartesian coordinates of the points using the Pythagorean theorem, therefore occasionally being called the Pythagorean distance. The distance between two objects that are not points is usually defined to be the smallest distance among pairs of points from the two objects. Formulas are known for computing distances between different types of objects, such as the distance from a point to a line.

“TLX3”, also known as T Cell Leukemia Homeobox 3, HOX11L2, RNX, T-Cell Leukemia Homeobox Protein 3, Homeobox Protein Hox-11L2, Homeo Box 11-Like 2, T-Cell Leukemia, Homeobox 3, or T-Cell Leukemia Homeobox 3, is an orphan homeobox protein that encodes a DNA-binding nuclear transcription factor.

The term “motoneuron” or “motor neuron” applies to neurons located in the central nervous system (CNS) that project their axons outside the CNS and directly or indirectly control muscles. Motor neuron is also synonymous with efferent neuron. According to their targets, motoneurons are classified into three broad categories: “Somatic motoneurons”, which directly innervate skeletal muscles, involved in locomotion (such as muscles of the limbs, abdominal, and intercostal muscles), “Special visceral motoneurons”, also called “branchial motoneurons”, which directly innervate branchial muscles (that motorize the gills in fish and the face and neck in land vertebrates) and “General visceral motoneurons”, also termed “visceral motoneurons”, which indirectly innervate smooth muscles of the viscera (e.g. the heart, and the muscles of the arteries). Visceral motoneurons synapse onto neurons located in ganglia of the autonomic nervous system (sympathetic and parasympathetic), located in the peripheral nervous system (PNS), which themselves directly innervate visceral muscles (and also some gland cells). All motoneurons are cholinergic, i.e. they release the neurotransmitter acetylcholine. Parasympathetic ganglionic neurons are also cholinergic, whereas most sympathetic ganglionic neurons are noradrenergic, releasing the neurotransmitter noradrenaline. Somatic motoneurons are further subdivided into two types: alpha efferent neurons and gamma efferent neurons. “Alpha motoneurons” innervate extrafusal muscle fibres (also termed muscle fibres) located throughout the muscle. “Gamma motoneurons” innervate intrafusal muscle fibres found within the muscle spindle. In addition to voluntary skeletal muscle contraction, alpha motoneurons also contribute to muscle tone. Gamma motoneurons regulate the sensitivity of the spindle to muscle stretching. Furthermore, alpha motoneurons can be further classified into the functional subtypes: fast-fatigable (FF), fast fatigue-resistant (FR) and slow(S) motoneurons, which show distinct excitability and recruitment properties and establish motor units (consisting of one motoneuron and all the muscle fibers it innervates) with markedly distinct fatigue and force properties (Burke, R.E. Physiology of motor units. in Myology (eds. Engel, A. G. & Franzini-Armstrong, C.) 464-484 (McGraw-Hill, New York, 1994)).

As used herein, “expression” includes but is not limited to one or more of the following: transcription of the gene into precursor mRNA; splicing and other processing of the precursor mRNA to produce mature mRNA; mRNA stability; translation of the mature mRNA into protein (including codon usage and mRNA availability); and glycosylation and/or other modifications of the translation product, if required for proper expression and function.

As used herein, the term “gene” means a segment of DNA that contains all the information for the regulated biosynthesis of an RNA product, including promoters, exons, introns, and other untranslated regions that control expression.

As used herein, the term “genotype” means an unphased 5′ to 3′ sequence of nucleotide pair(s) found at one or more polymorphic sites in a locus on a pair of homologous chromosomes in an individual. As used herein, genotype includes a full-genotype and/or a sub-genotype.

As used herein, the term “locus” means a location on a chromosome or DNA molecule corresponding to a gene or a physical or phenotypic feature.

As used herein, the term “isogene” means the different forms of a given gene that exist in the population.

As used herein, the term “mutant” means any heritable variation from the wild-type that is the result of a mutation, e.g., single nucleotide polymorphism. The term “mutant” is used interchangeably with the terms “marker”, “biomarker”, and “target” throughout the specification.

As used herein, the term “subject” means that preferably the subject is a mammal, such as a human, but can also be an animal, including but not limited to, domestic animals (e.g., dogs, cats and the like), farm animals (e.g., cows, sheep, pigs, horses and the like) and laboratory animals (e.g., monkeys such as cynomolgus monkeys, rats, mice, guinea pigs and the like). “Upregulation” and “downregulation” of gene expression can be assessed using statistical methods well known to the person skilled in the art.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

### Materials & Methods

### Mice

All animal procedures were approved by and carried out in accordance with guidelines of the Veterinary Department of the Canton Basel-Stadt, Switzerland. The mice used in this study were kept on a C57BL/6 background and were of the following genotype: 6 C57/Bl6 (Charles River Laboratories), 4 Emx1-Cre mice (Gorski et al., 2002), 4 Cux2-CreERT2 (Franco et al., 2012), 7 Scnn1a-Cre (Madisen et al., 2010), 15 Tlx3-Cre(PL56) (Gerfen et al., 2013), 3 Ntsr1-Cre (Gong et al., 2007), 2 PV-Cre (Hippenmeyer et al., 2005), 6 VIP-Cre (Taniguchi et al., 2011), 5 Sst-Cre (Taniguchi et al., 2011) were used for widefield calcium imaging. Ai148 ((Daigle et al., 2018), Jackson Laboratories, stock #030328) mice were used as breeders to drive GCaMP6f in Cre positive neurons. 7 C57BL/6 were used for experiments controlling for hemodynamic responses. To induce expression of Cre in the Cux2-CreERT2 line, mice were kept for at least 1 week on Tamoxifen containing food (Envigo, 400 mg Tamoxifen per kg of food) as their only food source. Mice were group housed in a vivarium (light/dark cycle: 12/12 hours). Experimental mice used were of both sexes.

### Surgery and Virus Injections

For all surgical procedures, mice were anesthetized using a mixture of Fentanyl (0.05 mg/kg; Actavis), Midazolam (5.0 mg/kg; Dormicum, Roche) and Medetomidine (0.5 mg/kg; Domitor, Orion). In a subset of mice (6 C57/Bl6, 4 Emx1-Cre, 4 Sst-Cre mice) we injected an AAV coated with the PHP.eb capsid (Chan et al., 2017) retroorbitally (6 ml each eye of at least 10¹³ GC/ml) to drive expression pan-neuronally or cell type specificly of GCaMP6 under either the Ef1α or hSyn promoter. For hemodynamic response control experiments, the inventors used an AAV PHP.eb Ef1α-GFP to pan-neuronally transfect GFP. To improve optical access to the cortex, they implanted crystal skull cranial windows (Kim et al., 2016). Prior to removing the skull plate overlying dorsal cortex, they zeroed bregma with a digital readout attached to the stereotax for later use as a landmark. Superglue (Pattex) was used to close the craniotomy. For the comparison of hemodynamic responses, the inventors did not remove the skull plate but instead glued the crystal skull coverslip directly onto the cleaned skull surface. A custom-machined titanium head bar was attached to the skull using dental cement (Paladur, Heraeus). An epifluorescence overview image was taken to mark reference points on the dorsal cortical surface. Anesthesia was antagonized by an intraperitoneal injection of a mixture of Flumazenil (0.5 mg/kg; Anexate, Roche) and Atipamezole (2.5 mg/kg; Antisedan, Orion Pharma), analgesia was achieved by injecting Buprenorphine (0.1 mg/kg; Reckitt Benckiser Healthcare (UK) Ltd.) and Metacam (5 mg/kg; Boehringer Ingelheim), and mice were subsequently returned to their home cage. Imaging commenced not before 1 week after headbar implantation and not before at least 3 weeks had passed in the cases in which AAVs had been used to transfect neurons. Clozapine (Sigma), Aripiprazole (Otsuka Pharmaceutical), Haloperidol (Janssen) and Amphetamine (Hänseler) were intraperitoneally injected at 0.2 mg/g, 0.2 mg/g, 0.1 mg/g and 4 mg/g body weight, respectively.

### Virtual Reality Setup and Stimulus Design

For all experiments we used a virtual reality setup as previously described (Leinweber et al., 2014). Mice were head-fixed and free to run on a spherical, air supported Styrofoam ball. Rotation of the ball was coupled to movement in a virtual reality environment. A virtual corridor was projected (Samsung SP-F10M) onto a toroidal screen positioned in front of the mice covering approximately 240 degrees horizontally and 100 degrees vertically of the field of view. Recordings usually consisted of 5 mins each in 4 sessions of a closed loop condition in which the translational movement of the Styrofoam ball was coupled to the mouse's own movement with the exception of mismatch stimuli, in which the visual flow was briefly halted for 1 s, 4 sessions of an open loop condition in which ball movement and mouse movement were decoupled and the visual stimulus consisted of a replay of the visual flow recorded in the previous closed loop condition, 2 sessions of a dark condition in which there was no visual stimulus and mice could locomote freely on the ball, and 2 sessions of a grating condition in which a gray screen to drifting grating stimulus (each parameter randomized: 8 cardinal orientations, mean duration 6 s±2 s SD, mean inter grating interval: 4.5 s±1.5 s SD) was shown. These sessions totaling approximately 1 hour recording time per day were acquired on each of 3 consecutive days. For mice in which the inventors tested the effect of drugs on dorsal cortex activity, the first post drug injection data was acquired +1 h after drug injection (+30 mins in the case of Amphetamine because of faster onset of the drug) and further data was collected at +24 h and +48 h. Data are shown as the combined data over either all data collected before drug injection (labelled as naïve) or after drug injection (labelled as Clozapine, Aripiprazole, Haloperidol or Amphetamine, respectively).

### Data Acquisition

All widefield imaging experiments were performed on a custom-built macroscope consisting of commercially available objectives mounted face to face (Nikon 85 mm/f1.8 sample side, Nikon 50 mm/f1.4 sensor side). The inventors used a 470 nm LED (Thorlabs) powered by a custom-built LED driver for exciting GCaMP (and GFP) fluorescence through an excitation filter (SP490, Thorlabs) and by means of a dichroic mirror (LP490, Thorlabs) placed in the parfocal plane of the objectives. Green fluorescence was collected through a 525/50 emission filter on an sCMOS camera (PCO edge 4.2). Apertures on objectives were usually kept wide open and the current at the LED driver was used to adjust fluorescence intensity to a value that was kept below 25% of the maximum dynamic range of the sensor. In cases where this was not possible (e.g. transfection of GFP yielded extremely bright fluorescence, often visible with the naked eye), objective apertures were gradually closed to avoid overexposure of the sensor. LED illumination was adjusted with a collimator (Thorlabs SM2F32-A) to achieve homogenous illumination across the cranial window surface. The resulting gaussian profile of the illumination cone was further trimmed with black tape on the sample side objective to avoid light shining directly into the mouse's eye. An Arduino board (Arduino Mega 2560) was used to control LED onsets slaved to the frame trigger signal of the camera. The duty cycle of the 470 nm LED was 90% for GCaMP imaging or 45% for GFP imaging in which case the LED power onset was also programmatically aligned to the core exposure time of the rolling shutter to avoid channel crossover artefacts. Raw images were acquired at full speed (100 Hz; except for GFP controls, which were acquired at 50 Hz effective frame rate) and full dynamic range (16 bit) of the sensor. The raw images were cropped on-sensor and the resulting data was streamed to disk with custom written software in LabVIEW (National Instruments), resulting in an effective pixel size of 6 mm² at a standardized imaging resolution of 1108 pixels×1220 pixels=1.35 Mpx.

### Data Processing

Raw movie data was manually registered across days by aligning subsequent mean projections of the data to the first recorded image sequence. The inventors placed regions of interest (ROIs) relative to readily identifiable anatomical landmarks, landmarks that had been previously noted during cranial window surgery and the average moving grating onset response as an additional guide to identify primary visual cortex relative to the landmarks. This resulted in the selection of six 20 px×20 px ROIs per hemisphere. Of those ROIs, they calculated the activity as the DF/F₀, wherein F₀ was the median fluorescence of the recording (approximately 30 000 frames in 5 mins recording time). DF/F₀ was corrected for slow fluorescence drift caused by thermal brightening of the LED using 8^{th} percentile filtering with a 62.5 s moving window akin to what was described previously for two-photon imaging (Dombeck et al., 2007).

### Data Analysis

Locomotion and visual flow onsets were determined as the time of crossing a threshold of 15 cm/s. The onset of drifting grating and closed loop visual flow halts (mismatches) were encoded by discrete voltage steps during data acquisition. Activity traces or image sequences were then aligned to those onset times.

The baseline subtraction window for unpredictable stimuli (mismatch and grating onsets) was −200 ms to 0 ms. For closed loop and open loop locomotion onsets, and open loop visual flow onsets, to accommodate calcium indicator offset dynamics and potential anticipatory activity, the inventors chose a window of 3 s prior to the time of threshold crossing during which the mouse would have to be sitting or no visual flow present. Baseline subtraction windows for these locomotion onsets and visual flow onsets were then accordingly placed at −2900 ms to −2700 ms relative to event onset. Varying this window within the bounds of offset dynamics of the indicator and anticipatory activity onset did not change their conclusions.

After antipsychotic drug injection, seizure-like activity was extremely rarely observed. To avoid these events distorting our conclusions, the inventors removed each 5 min recording from the data set in which the average activity DF/F of all 12 ROIs stayed elevated more than 30% for more than 10 s. This criterion led to the exclusion of 0.26% of data (10 out of 3801 of 5 min recordings).

To quantify the similarity of closed loop and open loop locomotion onset responses, the inventors calculated for the naïve data set Pearson's correlation coefficient for the averaged responses (onsets and mice) in a window −3 s to +3 s, but separately for each ROI.

To calculate the maps that show the timing of activation of individual dorsal cortex regions, they first aligned the craniotomies of all mice within one genotype to the first mouse's average grating onset response map and then calculated an average dorsal cortex response time course. For each pixel of this image sequence, the inventors then determined the time at which the pixel's value crossed a threshold of the maximum value in the entire time course divided by 6 standard deviations of the pixel's baseline value (−200 ms to 0 ms before event onset). The resulting maps were denoised using a Gaussian filter and they removed all pixel clusters that contained fewer than 500 connected pixels.

For correlation analyses, the inventors calculated Pearson's correlation coefficient between region's pairwise activity. They then determined the Euclidean distance of each ROI and normalized this value to the Euclidean distance between bregma and lambda obtained from the images during initial surgery. They then calculated the density of the resulting distribution of pairwise region's distance and correlation of activity in a 40 pt×40 pt grid and smoothed the resulting image with a gaussian filter to obtain heatmaps. Contour lines in the heatmaps were then drawn to 50% of the bin with the maximum value in the plot. Boxplot quantifications were calculated as the difference of a region pair's correlation coefficient of activity after and before treatment, normalized to the value before treatment and split into 2 groups by an arbitrary threshold of 0.9 of the bregma-lambda distance.

To obtain differences in correlation of activity and locomotion behavior before and after Clozapine injection, the inventors first averaged all correlation coefficients in the individual sessions of a condition to generate one correlation coefficient per mouse and ROI, for each condition. They then calculated and plotted the difference between these correlation values after and before drug injection.

### Results

### The Antipsychotic Drug Clozapine Alters Visuomotor Integration in Layer 5

In visual cortex, the activation of an internal representation is a mouse brain's best guess at what the stimuli in the environment are. The inventors speculated that the activation of such an internal representation would be particularly susceptible to drugs that are known to reduce or enhance illusory percepts. And if so, they should find that the primary effect in dorsal cortex of these drugs should also be found in internal representation neurons. The inventors thus quantified the changes in dorsal cortex associated with a single intraperitoneal injection of an antipsychotic drug (Clozapine). In C57/Bl6 mice that expressed GCaMP6 brain wide, they found that closed loop and open loop locomotion onsets were almost unaffected by Clozapine, while there was a small increase in open loop visual flow onset responses. In mice that expressed GCaMP6 in Tlx3 positive layer 5 neurons, however, Clozapine fundamentally changed both closed loop and open loop locomotion onset responses. Both types of locomotion onset resulted in a massive increase in activity. Conversely, open loop visual flow onset responses remained largely unchanged. Clozapine also had an opposing effect on the average correlation of activity with locomotion in Tlx3 positive layer 5 neurons: While average correlation of activity and locomotion was increased in C57/Bl6 mice that expressed GCaMP6 brain wide, the same measure was decreased in mice that expressed GCaMP6 in Tlx3 positive layer 5 IT neurons. Similarly, the inventors found that Clozapine increased mean activity in Tlx3 positive neurons but found no evidence of a change in mean activity in C57/Bl6 mice that expressed GCaMP6 brain wide. Thus, Clozapine exhibited a substantially stronger influence on locomotion onset responses in Tlx3 positive layer 5 IT neurons than would be expected from the effect of Clozapine on brain wide responses.

### Antipsychotics Decouple Long-range Cortico-cortical Activity

Excitatory layer 5 IT neurons are the primary source of long-range cortical communication and are one prominent source of locomotion related activity in V1. Thus, it is possible that Clozapine acts to reduce long-range influence between layer 5 IT neurons. To investigate this possibility, the inventors computed pairwise correlations of calcium activity between the six regions of interest across both hemispheres and all recording conditions. They did this first in C57/Bl6 mice that expressed GCaMP6 brain wide before and after a single intraperitoneal injection of Clozapine. Overall, Clozapine resulted in a relatively modest decrease in correlations. When repeating this experiment in mice that expressed GCaMP6 in Tlx3 positive layer 5 IT neurons, the inventors found a larger decrease in correlations that appeared to be stronger for areas that were further apart. To quantify these changes, they calculated the correlations as a function of linear distance between the areas as measured in a top view of dorsal cortex. To visualize the distribution of correlations, they interpolated the data using a heatmap. They then split the data into approximately equal portions of short and long-range correlations using a cutoff of 0.9 of the bregma-lambda distance (approx. 3.8 mm). While both short- and long-range correlations were reduced, this reduction was not significant for either of them. They also found no evidence of a difference between short- and long-range correlation changes. In mice that expressed GCaMP6 in Tlx3 positive layer 5 IT neurons, however, the inventors found a reduction in both short- and long-range correlations, and both were more strongly reduced than the reduction observed in C57/Bl6 mice that expressed GCaMP6 brain wide (C57/Bl6 vs Tlx3-Cre x Ai148, short-range: p<0.02; long-range: p<10^{-5.}; ranksum test). Moreover, the reduction in activity correlations was stronger in long-range correlations than it was in short-range correlations. To confirm that the Clozapine induced reduction in correlation was stronger in deep cortical layers, the inventors repeated the experiment in a population of layer 2/3 and layer 4 excitatory neurons using Cux2-CreERT2 x Ai148 mice. Consistent with the hypothesis that the strongest effect of Clozapine was observed in layer 5 IT neurons, they found that Clozapine decreased correlations of cortical activity also in superficial excitatory neurons, but this reduction was significantly weaker than the reduction we observed in mice that express Tlx3 positive layer 5 IT neurons (Cux2-CreERT2 x Ai148 vs Tlx3-Cre x Ai148, short-range: p<0.005, long-range: p<10⁻⁸, ranksum test). Thus, administration of the antipsychotic drug Clozapine resulted in a decorrelation of activity across dorsal cortex that was stronger in layer 5 IT neurons than it was in either layer 2/3 neurons or in the brain wide average.

To test whether the decorrelation of the activity of Tlx3 positive layer 5 IT neurons is specific to Clozapine or might more generally be a functional signature of antipsychotic drugs, the inventors repeated the experiments with two additional antipsychotic drugs, Aripiprazole and Haloperidol. Surprisingly, they found that the principal effect of decorrelation of the activity patterns of Tlx3 positive layer 5 IT neurons was preserved with both drugs. These changes in correlation were absent upon injection of the psychostimulant Amphetamine. This indicates that antipsychotics act by reducing the coupling strength of long-range interactions between cortical layer 5 IT neurons.

Changes in the correlation between cortical areas can either be driven by changes in common external inputs or through changes in the direct communication. Assuming the primary source of the antipsychotic drug driven change in correlation is a reduction of the strength of the direct communication between cortical areas, the reduction of the spread of signals that originate in one area of cortex would be expected. To test this, the inventors investigated how the spread of visuomotor prediction error responses in Tlx3 positive layer 5 IT neurons is influenced by antipsychotic drugs. Negative and positive visuomotor prediction error responses are thought to be computed in V1 from where they spread to secondary visual and associative areas of cortex (Jordan and Keller, 2020; Keller and Mrsic-Flogel, 2018). The computation of negative prediction errors during movement requires a prediction of visual flow, which is mediated by long-range cortical input from areas like A24b (Leinweber et al., 2017), as well as a bottom-up visual signal. By contrast, the computation of a positive prediction error during passive observation in principle only requires a bottom-up visual signal, assuming a prediction of no-change is signaled as an absence of activity in top-down input. Thus, a reduction in mismatch responses in Tlx3 neurons and a reduction in the spread of this signal to secondary visual areas was expected. This is indeed what the inventors observed: mismatch responses were partially reduced in V1 and almost absent in V2 am. Responses to onsets of moving gratings were also reduced in V2 am, but overall less affected by the antipsychotic drugs. These results support the interpretation that antipsychotic drugs reduce lateral communication in layer 5.

As is apparent to one of ordinary skill in the art, variations in the above-described methods can be introduced with ease to attain the same objective. Various incubating conditions, labels, apparatus and materials can be chosen according to individual preference. All publications referred to herein are incorporated by reference in their entirety as if each were referred to individually.

1. A method of screening for psychoactive agents comprising the steps of
a) simultaneously assessing in a subject, the activity of neurons of at least two regions of the deep cortical layers in the absence of a potential psychoactive agent and correlating these activities,
b) simultaneously assessing in said subject, the activity of said neurons of said at least two regions of the deep cortical layers in the presence of the potential psychoactive agent and correlating said activities, and
c) comparing the correlations obtained in step a) and b),
wherein any significant change in the correlation obtained in step b) as compared to the correlation obtained in step a) is indicative of a potential psychoactive activity of said potential psychoactive agent.

2. The method of claim 1 wherein said neurons of deep cortical layers are neurons of cortical layers 5 and/or 6.

3. The method of claim 1 wherein the second region is in an anatomically different area of the brain as compared to the first region.

4. The method of claim 1 wherein the activity of neurons of the deep cortical layers of at least six different regions of the brain is measured in the presence and in the absence of the potential psychoactive agent.

5. The method of claim 1 wherein the activities of the neurons are measured while the subject is performing a task.

6. The method of claim 5 wherein said task involves movement, for instance locomotion.

7. The method of claim 1 wherein a decrease in correlation can be indicative of an antipsychotic activity of said potential psychoactive agent.

8. The method of any of claim 1 wherein an increase in correlation can be indicative of a propsychotic activity of said potential psychoactive agent.

9. The method of claim 1 wherein the activity of the neurons is assessed by EEG, MRI, fMRI, Magnetoencephalography, widefield calcium imaging, or two photon imaging.

10. The method of claim 1 wherein the distance, for instance Euclidean distance, between the different brains regions is used in the assessment of the correlation between the change of activities measures in step a) and b).

11. The method of claim 1 wherein the neurons of the deep cortical layers are neurons with intratelencephalic projection patterns.

12. The method of claim 1 further comprising the steps of
d) simultaneously assessing in a subject, the activity of neurons of at least two other regions of the brain that are not regions of the deep cortical layers in the absence of a potential psychoactive agent and correlating these activities,
e) simultaneously assessing in said subject, the activity of said neurons of said at least two other regions of the brain that are not regions of the deep cortical layers in the presence of the potential psychoactive agent and correlating said activities, and
f) comparing the correlations obtained in step d) and e),
wherein the absence of any significant change in the correlation obtained in step e) as compared to the correlation obtained in step d) is indicative of a psychoactive activity of said potential psychoactive agent when there is a significant change in the correlation obtained in step b) as compared to the correlation obtained in step a) is indicative of a potential psychoactive activity of said potential psychoactive agent.

13. The method of claim 12 wherein said region which is not a deep cortical layer is a region in cortical layers 1, 2 and/or 3.

14. A psychoactive agent identified using the method according to claim 1.

15. (canceled)
